(19) United States
(12) Patent Application Publication
(10) Pub. No.: US 2021/0047229 A1
(43) Pub. Date: Feb. 18, 2021

(54) GAS DETECTION MATERIAL

(71) Applicants: NIPPON ELECTRIC GLASS CO., LTD., Otsu-shi, Shiga (JP); TOHOKU INSTITUTE OF TECHNOLOGY, Sendai-shi, Miyagi (JP)

(72) Inventors: Masato TSUJIGUCHI, Otsu-shi (JP); Yasuko MARUO, Sendai-shi (JP)

(21) Appl. No.: 17/042,249

(22) PCT Filed: Jul. 1, 2019
(86) PCT No.: PCT/JP2019/026072

§ 371 Date: Sep. 28, 2020

(30) Foreign Application Priority Data
Jul. 13, 2018 (JP) 2018-132856
Feb. 6, 2019 (JP) 2019-019353

(51) Int. Cl. C03C 3/06; C07C 47/58; C03C 3/089

(52) U.S. Cl. CPC C03C 3/06; C03C 3/089; C07C 47/58

(57) ABSTRACT

Provided is a small-size, inexpensive gas detection material capable of detecting aldehyde-based gas. The gas detection material includes: a porous body having pores; an alkaline compound carried inside the pores; and a gas sensing agent carried inside the pores.

### TECHNICAL FIELD

The present invention relates to gas detection materials.

### BACKGROUND ART

Lung cancer is a type of cancer with highest mortality. The reason for this is that it is difficult to detect lung cancer early by chest roentgenography which is currently a major testing method for lung cancer. To cope with this, consideration is being given to a method for diagnosing lung cancer early by analyzing the expired breath of a subject for a component increasing specifically in the breaths of lung cancer patients.

For example, there is disclosed a result report that when the expired breaths of lung cancer patients were analyzed with a gas chromatography mass spectroscopy analyzer (GC-MS), their expired breaths contained high concentrations of aldehyde-based gas, such as nonanal, as compared to the expired breaths of healthy subjects (see, for example, Non-Patent Literature 1).

### CITATION LIST

### [Non-Patent Literature]

### [NPTL 1]

HANDA and MIYAZAWA, Detection of lung cancer using exhaled breath analysis, Journal of Clinical and Experimental Medicine, Vol. 240, No. 11, 933-935 (2012)

### SUMMARY OF INVENTION

### [Technical Problem]

However, the GC-MS has a large size, is very expensive, and has a problem that it takes a long time to analyze.

The present invention has been made in view of the foregoing circumstances and therefore has an object of providing a small-size, inexpensive gas detection material capable of detecting aldehyde-based gas.

### [Solution to Problem]

A gas detection material according to the present invention includes: a porous body having pores; an alkaline compound carried inside the pores; and a gas sensing agent carried inside the pores. When the gas sensing agent carried inside the pores of the porous body reacts with aldehyde-based gas in the presence of the alkaline compound serving as a catalyst, the light absorbance of the gas sensing agent at a particular wavelength changes. By measuring this change in light absorbance, the aldehyde-based gas can be detected.

In the gas detection material according to the present invention, the porous body is preferably a porous glass containing, in terms of % by mass, 85 to 100% SiO₂.

In the gas detection material according to the present invention, the alkaline compound preferably contains sodium hydroxide.

In the gas detection material according to the present invention, the gas sensing agent preferably contains vanillin and/or a vanillin derivative.

The gas detection material according to the present invention is preferably for use in detecting aldehyde-based gas.

### [Advantageous Effects of Invention]

The present invention enables provision of a small-size, inexpensive gas detection material capable of detecting aldehyde-based gas.

### DESCRIPTION OF EMBODIMENTS

A description will be given of a gas detection material according to the present invention.

The gas detection material according to the present invention includes: a porous body having pores; an alkaline compound; and a gas sensing agent. Both the alkaline compound and the gas sensing agent are carried inside the pores of the porous body.

Hereinafter, the description is given according to each component element.

### (Porous Body)

Since, as described previously, aldehyde-based gas is detected by measuring a change in the light absorbance of the gas sensing agent at a particular wavelength, the porous body is required to have a high light transmittance. Therefore, the porous body is preferably a porous glass having a high light transmittance. Alternatively, although inferior in light transmittance to the porous glass, a porous polymeric material, porous ceramics, silica gel or the like may be used as the porous body.

The description below is given of a method for producing a porous glass.

First, a glass base material for a porous glass is prepared in the following manner. Glass raw materials are formulated to give a glass composition containing, in terms of % by mass, 40 to 80% SiO₂, over 0 to 40% B₂O₃, over 0 to 20% Na₂O, 0 to 10% ZrO₂, 0 to 5% Al₂O₃, and 0 to 20% RO (where R represents at least one selected from among Mg, Ca, Sr, and Ba) and having a mass ratio of Na₂O/B₂O₃ of 0.25 to 0.5. The following description is given of the reasons why the content of each component is specified as above. In the following description of the respective contents of components, “%” refers to “% by mass” unless otherwise specified.

SiO₂ is a component that forms a glass network. The content of SiO₂ is 40 to 80%, preferably 45 to 75%, more preferably 50 to 70%, and particularly preferably 52 to 65%. If the content of SiO₂ is too small, the weather resistance and mechanical strength tend to decrease. On the other hand, if the content of SiO₂ is too large, phase separation is less likely to occur.

B₂O₃ is a component that forms a glass network and promotes phase separation. The content of B₂O₃ is over 0 to 40%, preferably 10 to 30%, and particularly preferably 20 to 25%. If the glass base material is free of B₂O₃, the above effects are difficult to achieve. On the other hand, if the content of B₂O₃ is too large, the weather resistance is likely to decrease.

The ratio B₂O₃/SiO₂ is preferably 0.3 to 0.5, more preferably 0.35 to 0.48, still more preferably 0.38 to 0.46, and particularly preferably 0.4 to 0.45. If the ratio B₂O₃/SiO₂ is too small, internal stress is likely to be generated in a later-described process for removing SiO₂ colloid with an aqueous alkaline solution, so that the porous glass is likely to crack. On the other hand, if the ratio B₂O₃/SiO₂ is too large, the mechanical strength is likely to decrease in the later-described process for removing SiO₂ colloid with an aqueous alkaline solution, so that the porous glass is likely to crack. The ratio “B₂O₃/SiO₂” represents the value obtained by dividing the content of B₂O₃ by the content of SiO₂.

Na₂O is a component that decreases the melting temperature to improve meltability and also a component that promotes phase separation. The content of Na₂O is over 0 to 20%, preferably 3 to 10%, and particularly preferably 4 to 8%. If the glass base material is free of Na₂O, the above effects are difficult to achieve. On the other hand, if the content of Na₂O is too large, phase separation is less likely to occur contrariwise.

The ratio Na₂O/B₂O₃ is 0.25 to 0.5, preferably 0.28 to 0.4, and particularly preferably 0.3 to 0.35. If the ratio Na₂O/B₂O₃ is too small, a boron oxide-rich phase is difficult to remove in a later-described process for removing the boron oxide-rich phase with an acid. On the other hand, if the ratio Na₂O/B₂O₃ is too large, the amount of expansion due to hydration of silica gel is likely to be smaller than the amount of contraction due to elution of Na₂O from a silica-rich phase, so that the porous glass is likely to crack.

ZrO₂ is a component that increases the weather resistance. If the porous glass reacts with an alkaline compound, the alkaline compound is consumed for the reaction and the amount of alkaline compound carried inside the porous glass is reduced, so that the function of the gas detection material may decrease. To cope with this, when the porous glass contains ZrO₂, the alkali resistance of the porous glass increases, so that the above inconvenience is less likely to occur. The content of ZrO₂ is 0 to 10%, preferably 1 to 10%, more preferably over 3 to 10%, still more preferably 4 to 8%, and particularly preferably 5 to 7%. If the content of ZrO₂ is too large, devitrification is likely to occur and phase separation is less likely to occur.

Al₂O₃ is a component that increases the weather resistance and mechanical strength. The content of Al₂O₃ is 0 to 5%, preferably 2 to 5%, and particularly preferably 3 to 4%. If the content of Al₂O₃ is too large, phase separation is less likely to occur.

RO (where R represents at least one selected from among Mg, Ca, Sr, and Ba) is a component that increases the content of ZrO₂ in a silica-rich phase and increases the weather resistance. The content of RO (i.e., the total content of MgO, CaO, SrO, and BaO) is 0 to 20%, preferably 0.5 to 20%, more preferably 1 to 17%, still more preferably 3 to 15%, yet still more preferably 4 to 13%, and particularly preferably 5 to 10%. If the content of RO is too large, phase separation is less likely to occur. The content of each of MgO, CaO, SrO, and BaO is 0 to 20%, preferably 1 to 17%, more preferably 3 to 15%, still more preferably 4 to 13%, and particularly preferably 5 to 10%. Of these, CaO is preferably used in view of its particularly large effect of increasing the weather resistance.

The glass base material for a porous glass according to the present invention may contain, in addition to the above components, the following components.

K₂O is a component that decreases the melting temperature to improve meltability and also a component that promotes phase separation. The content of K₂O is preferably 0 to 20%, more preferably 3 to 10%, and particularly preferably 4 to 8%. If the content of K₂O is too large, phase separation is less likely to occur contrariwise.

ZnO is a component that increases the content of ZrO₂ in a silica-rich phase and increases the weather resistance. The content of ZnO is preferably 0 to 20%, more preferably 0 to 10%, and particularly preferably 0 to below 3%. If the content of ZnO is too large, phase separation is less likely to occur.

The glass base material may contain, in addition to the above components, various components in a range not impairing the effects of the invention. For example, the glass base material may contain TiO₂, La₂O₃, Ta₂O₅, TeO₂, Nb₂O₅, Gd₂O₃, Y₂O₃, Eu₂O₃, Sb₂O₃, SnO₂, P₂O₅, and Bi₂O₃, each preferably in a range of 15% or less, more preferably 10% or less, particularly preferably 5% or less, and in a range of 30% or less in total content.

Next, the glass batch obtained by the formulation is melted at 1300 to 1500° C. for 4 to 12 hours. Subsequently, the molten glass is formed into a platy shape and then annealed at 400 to 600° C. for 10 minutes to 10 hours, thus obtaining a glass base material. The shape of the obtained glass base material is not particularly limited, but is preferably a platy shape having a rectangular or circular surface figure. In order to make the obtained glass base material into a desired shape, the glass base material may be subjected to processing, such as cutting or polishing. Furthermore, the glass base material may be continuously produced in a refractory furnace. The method for melting the glass and the method for forming the glass are not limited to the above methods.

The obtained glass base material preferably has an aspect ratio of 2 to 1000 and particularly preferably 5 to 500. If the aspect ratio is too small, this creates a large difference in the rate of removal of a boron oxide-rich phase between the surface and inside of the glass base material in a later-described process for removing the boron oxide-rich phase with an acid, so that stress is likely to be generated and the porous glass is therefore likely to crack. On the other hand, if the aspect ratio is too large, the glass base material is difficult to handle. The aspect ratio can be calculated by the following equation.

Aspect ratio=(base area of the glass base material)^{1/2}/(thickness of the glass base material)

The base area and thickness of the obtained glass base material may be appropriately adjusted to give the above aspect ratio. For example, the base area is preferably 1 to 1000 mm² and particularly preferably 5 to 500 mm² and the thickness is preferably 0.1 to 1 mm and particularly preferably 0.2 to 0.5 mm.

Next, the obtained glass base material is thermally treated to separate it into two phases: a silica-rich phase and a boron oxide-rich phase. The thermal treatment temperature is preferably 500 to 800° C. and particularly preferably 600 to 700° C. If the thermal treatment temperature is too high, the glass base material softens and is therefore less likely to obtain a desired shape. On the other hand, if the thermal treatment temperature is too low, the glass base material is less likely to undergo phase separation. The thermal treatment time is preferably ten minutes or more, more preferably an hour or more, and particularly preferably three hours or more. If the thermal treatment time is too short, the glass base material is less likely to undergo phase separation. The upper limit of the thermal treatment time is not particularly limited. However, even if the glass base material is thermally treated for a long time, phase separation does not progress beyond a certain level. Therefore, the thermal treatment time is actually not more than 180 hours.

Next, the glass base material separated into two phases is immersed into an acid to remove the boron oxide-rich phase, thus obtaining a porous glass. The acid that can be used is hydrochloric acid or nitric acid. These acids may be used in mixture. The concentration of the acid is preferably 0.1 to 5 N and particularly preferably 0.5 to 3 N. The time for immersion in the acid is preferably an hour or more, more preferably 10 hours or more, and particularly preferably 20 hours or more. If the time for immersion is too short, a porous glass is less likely to be obtained. The upper limit of the time for immersion is not particularly limited, but it is actually not more than 100 hours. The temperature during immersion is preferably 20° C. or higher, more preferably 25° C. or higher, and particularly preferably 30° C. or higher. If the temperature during immersion is too low, a porous glass is less likely to be obtained. The upper limit of the temperature during immersion is not particularly limited, but it is actually not higher than 95° C.

In the process for thermally treating the glass base material to separate it into two phases: a silica-rich phase and a boron oxide-rich phase, a silica-containing layer (a layer containing silica in a content of approximately 80% by mass or more) tends to be formed in the uppermost portion of the surface of the glass base material. The silica-containing layer is difficult to remove with an acid. Therefore, if a silica-containing layer has been formed, the glass base material separated into phases is cut and polished to remove the silica-containing layer and then immersed into an acid. Thus, the boron oxide-rich phase can be more easily removed.

Furthermore, it is preferred to remove residual ZrO₂ colloid and SiO₂ colloid in the pores of the obtained porous glass. The following description is given of a method for removing ZrO₂ colloid and a method for removing SiO₂ colloid. However, the methods are not limited to the following.

ZrO₂ colloid can be removed, for example, by sulfuric acid. The concentration of sulfuric acid is preferably 0.1 to 5 N and particularly preferably 1 to 5 N. The time for immersion in sulfuric acid is preferably an hour or more and particularly preferably 10 hours or more. If the time for immersion is too short, ZrO₂ colloid is less likely to be removed. The upper limit of the time for immersion is not particularly limited, but it is actually not more than 100 hours. The temperature during immersion is preferably 20° C. or higher, more preferably 25° C. or higher, and particularly preferably 30° C. or higher. If the temperature during immersion is too low, ZrO₂ colloid is less likely to be removed. The upper limit of the temperature during immersion is not particularly limited, but it is actually not higher than 95° C.

SiO₂ colloid can be removed, for example, by an aqueous alkaline solution. Examples of the alkali that can be used include sodium hydroxide and potassium hydroxide. These alkalis may be used in mixture. The time for immersion in the aqueous alkaline solution is preferably 10 minutes or more and particularly preferably 30 minutes or more. If the time for immersion is too short, SiO₂ colloid is less likely to be removed. The upper limit of the time for immersion is not particularly limited, but it is actually not more than 100 hours. The temperature during immersion is preferably 15° C. or higher and particularly preferably 20° C. or higher. If the temperature during immersion is too low, SiO₂ colloid is less likely to be removed. The upper limit of the temperature during immersion is not particularly limited, but it is actually not higher than 95° C.

The obtained porous glass preferably contains, in terms of % by mass, 85 to 100% SiO₂. The porous glass may contain Al₂O₃, ZrO₂, and so on as other components.

The median diameter of the pore distribution of the porous glass is preferably 1 to 100 nm, more preferably 4 to 90 nm, and particularly preferably 7 to 80 nm. If the median diameter of the pore distribution is too small, gas diffusion into the pores is significantly difficult. On the other hand, if the median diameter of the pore distribution is too large, the light transmittance tends to decrease. The pores have various shapes, such as spherical, approximately ellipsoidal, and tubular shapes. The thickness and aspect ratio of the porous glass are the same as those of the glass base material.

The light transmittance of the porous glass with a thickness of 0.5 mm at a wavelength of 400 nm is preferably 0.02% or more, more preferably 0.05% or more, and particularly preferably 0.1% or more. If the light transmittance is too low, the porous glass tends to be difficult to use as a porous body for a gas detection material.

### (Alkaline Compound)

Examples of the alkaline compound that can be used include hydroxides of alkali metals, carbonates of alkali metals, hydrogencarbonates of alkali metals, hydroxides of alkaline earth metals, and carbonates of alkaline earth metals. Of these, sodium hydroxide, which has high catalytic power, is preferably used.

### (Gas Sensing Agent)

There is no particular limitation as to the type of gas sensing agent for use so long as it has an absorption wavelength of 350 to 750 nm and reacts with aldehyde-based gas in the presence of an alkaline compound to change its light absorbance, but vanillin and/or a vanillin derivative are preferably used. Vanillin and/or vanillin derivatives have the advantage of being easy to handle because they do not volatilize at room temperature. Alternatively, other gas sensing agents can also be used.

Next, a description will be given of an example of a method for producing the gas detection material according to the present invention.

First, an alkaline compound and a gas sensing agent are mixed with a solvent, such as water, thus obtaining a mixed liquid containing the alkaline compound and the gas sensing agent. The concentration of the alkaline compound in the mixed liquid is preferably 0.1 to 10 N and particularly preferably 0.25 to 5 N. If the concentration of the alkaline compound is too low, the reaction between gas and the gas sensing agent may not sufficiently progress. On the other hand, if the concentration of the alkaline compound is too high, the alkaline compound is likely to react with a porous body, so that the mechanical strength of the porous body may decrease.

The amount of the gas sensing agent added (i.e., the content thereof in the mixed liquid) is, in terms of mass ratio relative to a porous body, preferably (gas sensing agent)/(porous body)=0.01 to 100 and particularly preferably 0.1 to 10. If the amount of the gas sensing agent added is too small, the function of the gas detection material tends to be insufficient. On the other hand, if the gas sensing agent is too much, it may fill the pores of the porous body, also in which case the function of the gas detection material tends to be insufficient.

Next, a porous body is immersed into the obtained mixed liquid, thus obtaining a gas detection material in which the alkaline compound and the gas sensing agent are carried inside the pores of the porous body. A porous body weighing 0.01 g to 10 kg (preferably 10 g to 10 kg) is preferably immersed into 0.01 to 100 L (preferably 0.1 to 10 L) of the mixed liquid and the time for immersion is preferably one minute to 50 hours. After the porous body is immersed into the mixed liquid, water therein may be volatilized by natural drying or other methods.

The alkaline compound and the gas sensing agent may be carried in the porous body by immersing the porous body into the mixed liquid multiple times. In this case, in a later stage of the immersion process, it is preferred to make the concentration of the alkaline compound in the mixed liquid higher. Thus, it can be more certainly prevented that in earlier stages of the immersion process a reaction product (for example, a silicic acid hydrate) of the alkaline compound and the porous body blocks the carrying of the gas sensing agent. For example, it is preferred to first immerse the porous body into a mixed liquid containing the alkaline compound at a concentration of 0.01 to 0.5 N and the gas sensing agent and then immerse the porous body into a mixed liquid containing the alkaline compound at a concentration of 0.1 to 10 N and the gas sensing agent.

Alternatively, the alkaline compound and the gas sensing agent may be separately carried in the porous body. Specifically, the gas sensing agent is mixed with a dispersion medium, such as water, thus obtaining a dispersant of the gas sensing agent. The porous body is immersed into the obtained dispersant, thus obtaining the porous body containing the gas sensing agent carried therein. Subsequently, the alkaline compound is mixed with a solvent, such as water, thus obtaining a solution of the alkaline compound. The porous body containing the gas sensing agent carried therein is immersed into the obtained solution, thus obtaining a gas detection material in which the gas sensing agent and the alkaline compound are carried. Thus, the effect of more certainly preventing a reaction product of the porous body and the alkaline compound from blocking the carrying of the gas sensing agent can be obtained.

Next, a description will be given of a method for detecting aldehyde-based gas.

First, the light absorbance of the gas detection material at a particular wavelength is measured with a spectro-photometer or other means.

Next, the gas detection material is put into a Tetra Pak or the like in which a gas to be measured is encapsulated, and allowed to stand for one minute to five hours to expose the gas detection material to the gas to be measured. In order to promote the reaction between the gas detection material and the gas to be measured, the gas detection material after being exposed to the gas may be heated at 50 to 200° C. for five minutes to an hour.

Subsequently, the light absorbance of the exposed gas detection material at the particular wavelength is measured with the spectro-photometer or other means. If the measured light absorbance is different from the previously measured light absorbance of the gas detection material, this means that the gas to be measured contains aldehyde-based gas. If a calibration curve is previously made using a standard gas containing a known amount of aldehyde-based gas, it is possible to determine the amount of aldehyde-based gas from a difference in the light absorbance of the gas detection material between before and after the exposure.

### EXAMPLES

Hereinafter, the present invention will be described with reference to examples, but is not limited to these examples.

### Example 1

### (Production of Porous Glass)

First, raw materials formulated to give a glass composition containing, in terms of % by mass, 53% SiO₂, 23% B₂O₃, 7% Na₂O, 6% ZrO₂, 3% Al₂O₃, and 8% CaO were put into a platinum crucible and then melted therein at 1400° C. for six hours. During melting of the glass batch, molten glass was stirred using a platinum stirrer to homogenize it. Next, the molten glass was poured onto a carbon sheet to form it into a platy shape and then annealed at 500° C. for 30 minutes, thus obtaining a glass base material.

The obtained glass base material was thermally treated in an electric furnace at 675° C. for 72 hours to separate it into phases. The glass base material separated into phases was cut and polished to a size of 5 mm×5 mm×0.5 mm (thickness). Next, the glass base material was immersed into 1 N nitric acid (at 90° C.) for 48 hours and then immersed into 3 N sulfuric acid (at 90° C.) for 48 hours. Thereafter, the glass base material was washed with ion-exchange water, thus obtaining a porous glass.

When the surface of the obtained porous glass was observed with an FE-SEM (SU-8220 manufactured by Hitachi, Ltd.), the glass had a skeleton structure based on spinodal phase separation. The composition of the obtained porous glass was, in terms of % by mass, 93% SiO₂, 4% ZrO₂, and 3% Al₂O₃ and the median diameter of the pore distribution was 80 nm. The light transmittance of the porous glass with a thickness of 0.5 mm at a wavelength of 400 nm was 0.1%.

The composition was measured with an energy dispersive X-ray analyzer (EX-250 manufactured by Horiba, Ltd.).

The median value of the pore distribution was measured with a pore distribution measurement device (QUADRASORB SI manufactured by Quantachrome Instruments).

The light transmittance was measured with a spectro-photometer (UV-3100 manufactured by Shimadzu Corporation).

### (Production of Gas Detection Material)

First, 0.35 g of vanillin and each of 1 g, 4 g, 10 g, and 20 g of sodium hydroxide were mixed with 100 ml of pure water, thus obtaining four types of mixed liquids having respective sodium hydroxide concentrations of 0.25 N, 1 N, 2.5 N, and 5 N.

Next, 10 g of the porous glass was immersed into 100 ml of each obtained mixed liquid for two hours and then allowed to stand in the atmosphere for 24 hours to volatilize water, thus obtaining gas detection materials.

### (Detection of Nonanal)

First, when the gas detection material produced using the mixed liquid having a sodium hydroxide concentration of 5 N was measured in terms of light absorbance at a wavelength of 420 nm with a spectro-photometer (UV-3100 manufactured by Shimadzu Corporation), the light absorbance (a. u.) was 4.2.

Next, the gas detection material was put into a Tetra Pak in which a gas containing 2.5 ppm of nonanal was encapsulated, and allowed to stand for four hours to expose the gas detection material to the gas. Subsequently, the exposed gas detection material was heated at 100° C. for 20 minutes.

When the heated gas detection material was measured in terms of light absorbance at a wavelength of 420 nm with the spectro-photometer, the light absorbance increased to 4.5 which was 0.3 higher than the light absorbance before the exposure. When the gas detection materials produced using the mixed liquids having respective sodium hydroxide concentrations of 0.25 N, 1 N, and 2.5 N underwent the same test, their light absorbances after the exposure to the gas increased about 0.1 relative to those before the exposure. Thus, it was found that the gas detection material can detect nonanal on the order of ppm.

### Example 2

### (Production of Porous Glass)

A glass base material produced in the same manner as in Example 1 was thermally treated in an electric furnace at 675° C. for 36 hours to separate it into phases. The glass base material separated into phases was cut, polished, treated with an acid, and washed with ion-exchange water in the same manner as in Example 1, thus obtaining a porous glass. The obtained porous glass had a skeleton structure based on spinodal phase separation and the median diameter of the pore distribution was 50 nm. The light transmittance of the porous glass with a thickness of 0.5 mm at a wavelength of 400 nm was 1%.

### (Production of Gas Detection Material)

An amount of 0.35 g of vanillin and 2 g of sodium hydroxide were mixed with 100 ml of pure water, thus obtaining a mixed liquid having a sodium hydroxide concentration of 0.5 N. Separately, 20 g of sodium hydroxide was mixed with 100 ml of pure water, thus obtaining 5 N sodium hydroxide solution.

An amount of 1 g of the porous glass was first immersed into 100 ml of the mixed liquid having a sodium hydroxide concentration of 0.5 N for two hours, then allowed to stand in the atmosphere for 24 hours to volatilize water, and further immersed into 2.5 N sodium hydroxide solution for two hours.

Thereafter, the porous glass was allowed to stand in the atmosphere for 24 hours to volatilize water, thus obtaining a gas detection material.

### (Detection of Nonanal)

First, when the gas detection material was measured in terms of light absorbance at a wavelength of 420 nm with a spectro-photometer (UV-3100 manufactured by Shimadzu Corporation), the light absorbance was 3.1.

Next, the gas detection material was put into a Tetra Pak in which a gas containing 2.5 ppm of nonanal was encapsulated, and allowed to stand for four hours to expose the gas detection material to the gas. Subsequently, the exposed gas detection material was heated at 100° C. for 20 minutes.

When the heated gas detection material was measured in terms of light absorbance at a wavelength of 420 nm with the spectro-photometer, the light absorbance increased to 3.6 which was 0.5 higher than the light absorbance before the exposure. Thus, it was found that the gas detection material can detect nonanal on the order of ppm.

### INDUSTRIAL APPLICABILITY

The gas detection material according to the present invention is suitable for a wide range of applications, including expired breath diagnosis, skin-gas measurement, a breath checker, environmental monitoring, and working environment management.

1. A gas detection material comprising: a porous body having pores; an alkaline compound carried inside the pores; and a gas sensing agent carried inside the pores.

2. The gas detection material according to claim 1, wherein the porous body is a porous glass containing, in terms of % by mass, 85 to 100% SiO₂.

3. The gas detection material according to claim 1, wherein the alkaline compound is sodium hydroxide.

4. The gas detection material according to claim 1, wherein the gas sensing agent is vanillin and/or a vanillin derivative.

5. The gas detection material according to claim 1, being for use in detecting aldehyde-based gas.
